(19) United States
(12) Patent Application Publication
(10) Pub. No.: US 2014/0358057 A1
(43) Pub. Date: Dec. 4, 2014

(54) Toe Walker Orthosis

(71) Applicant: Certified Prosthetic & Orthotics, LLC, Greeley, CO (US)

(72) Inventor: Jason L. Bradshaw, Eaton, CO (US)

(21) Appl. No.: 13/904,938

(22) Filed: May 29, 2013

(51) Int. Cl. A61F 5/01

(52) U.S. Cl. CPC A61F 5/0127
USPC 602/28

(57) ABSTRACT

An orthosis worn on the human foot to restrict foot flexion to correspondingly limit the ability of the orthosis wearer to walk on the toes.

### I. FIELD OF THE INVENTION An orthosis worn on the human foot to restrict foot flexion to correspondingly limit the ability of an orthosis wearer to walk on the toes.

### II. BACKGROUND OF THE INVENTION

The human foot and ankle is typically divided into a hindfoot, a midfoot, and a forefoot. The hindfoot is composed of the talus or ankle bone and the calcaneus or heel bone. The two long bones of the lower leg, the tibia and fibula, are connected to the top of the talus to form the ankle of the foot. Connected to the talus at the subtalar joint, the calcaneus, the largest bone of the foot, is cushioned inferiorly by a layer of fat. The five irregular bones of the midfoot, the cuboid, navicular, and three cuneiform bones, form the arches of the foot which serves as a shock absorber. The midfoot is connected to the hind- and fore-foot by muscles and the plantar fascia. The forefoot is composed of five toes and the corresponding five proximal long bones forming the metatarsus. Both the midfoot and forefoot constitute the dorsum (the area facing upwards while standing) and the planum (the area facing downwards while standing). The instep is the arched part of the top of the foot between the toes and the ankle.

Walking on the toes of the foot, also known as toe walking, is fairly common in children who are just beginning to walk; although it can occur in adults. Idiopathic toe walking may be treated by wearing an orthosis either during the day, night or both which limits the ability of the toe walker to walk on the toes and stretches the achilles tendon.

### III. SUMMARY OF THE INVENTION

Accordingly, a broad object of the invention can be to provide an orthosis which can be worn on the human foot to limit foot flexion and in particular plantar flexion to correspondingly limit the ability of the orthosis wearer to walk on the toes.

Another broad object of the invention can be to provide in a unitary or one-piece orthosis which limits the ability of the wearer to walk on the toes including a lower leg section which engages the lower leg and a plantar section which engages the foot.

Another broad object of the invention can be to provide an orthosis which limits the ability of the orthosis wearer to walk on the toes including a lower leg section discrete from a plantar section. The plantar section configured to be received in nested engagement within the lower leg section. The lower leg section further including a retention element operable in relation to the plantar section while in nested engagement within the lower leg section to retain the foot of the wearer within the orthosis to limit the ability of the wearer to walk on the toes.

Another broad object of the invention can be to provide a shank element which upwardly extends in relation to the plantar section to support a posterior leg intercept element which engages the posterior lower leg of said wearer at a location above the ankle and an below the soleus muscle and over the achilles tendon which acts to further limit flexion of the foot at the ankle and provide stimuli allowing conscious or unconscious perception of movement and spatial orientation of the foot.

Naturally, further objects of the invention are disclosed throughout other areas of the specification, drawings, photographs, and claims.

### IV. BRIEF DESCRIPTION OF THE DRAWINGS

FIG. 1 is a perspective view of a particular embodiment of the inventive orthosis having a posterior leg section which nests within a plantar section and worn on the human foot to restrict foot flexion and correspondingly limit the ability of the orthosis wearer to walk on the toes.

FIG. 2 is a perspective view of a particular embodiment of the inventive orthosis having a unitary posterior leg section and plantar section worn on the human foot to restrict foot flexion and correspondingly limit the ability of the orthosis wearer to walk on the toes.

FIG. 3 is an exploded view of the particular embodiment of the inventive orthosis shown in FIG. 1.

FIG. 4 is a first side view of the particular embodiment of the inventive orthosis shown in FIG. 1.

FIG. 5 is a second side view of the particular embodiment of the inventive orthosis shown in FIG. 1.

FIG. 6 is a front end view of the particular embodiment of the inventive orthosis shown in FIG. 1.

FIG. 7 is rear end view of the particular embodiment of the inventive orthosis shown in FIG. 1.

FIG. 8 is a top view of the particular embodiment of the inventive orthosis shown in FIG. 1.

FIG. 9 is a bottom view of the particular embodiment of the inventive orthosis shown in FIG. 1.

FIG. 10 is a exploded view which illustrates a method of making a particular embodiment of the inventive orthosis.

### V. DETAILED DESCRIPTION OF THE INVENTION

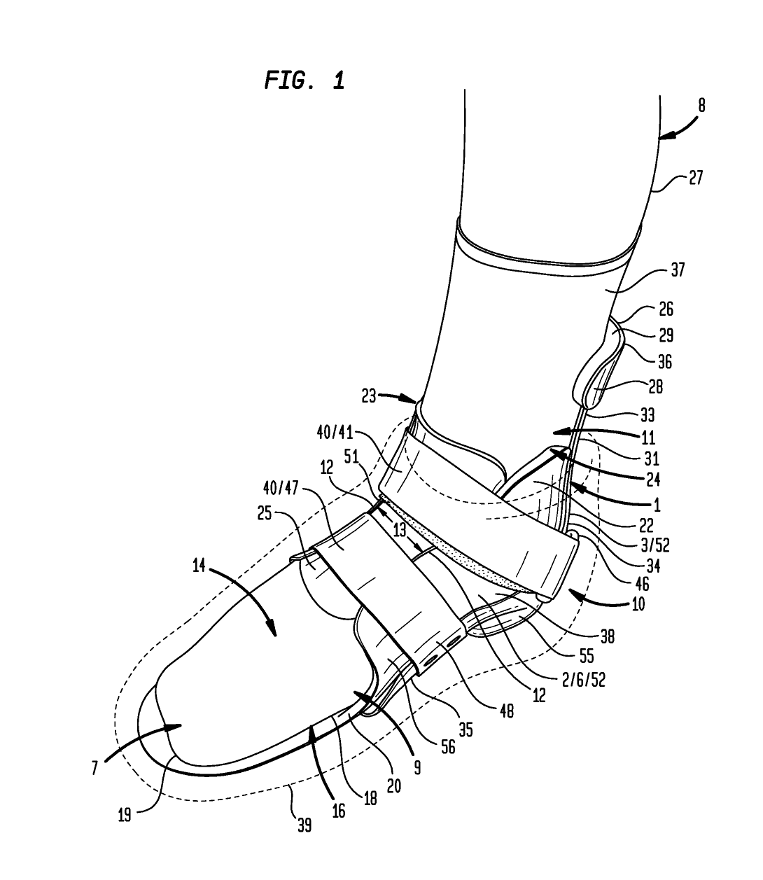

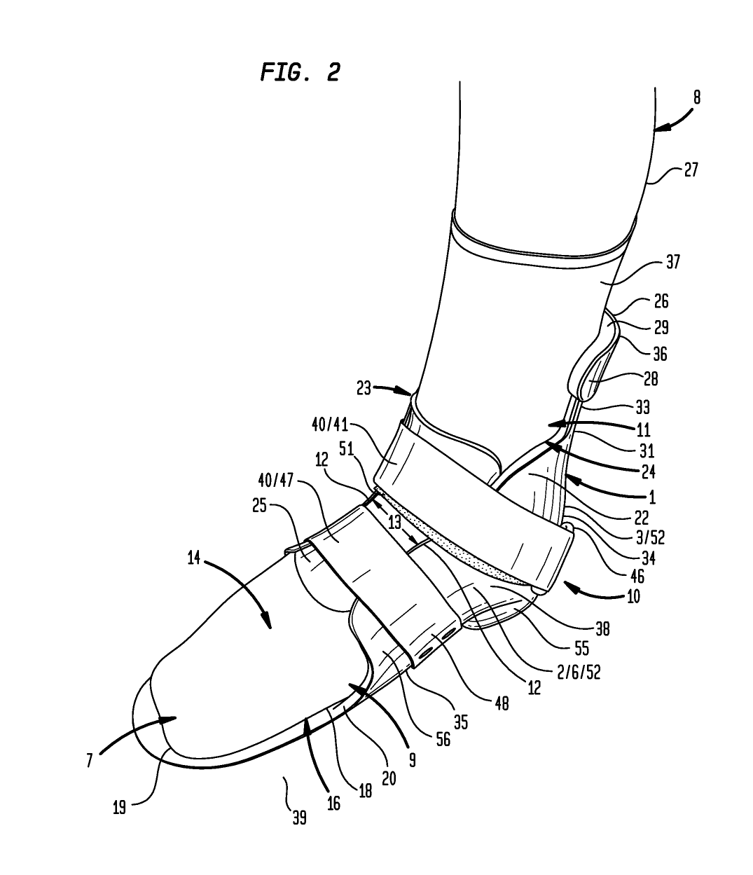

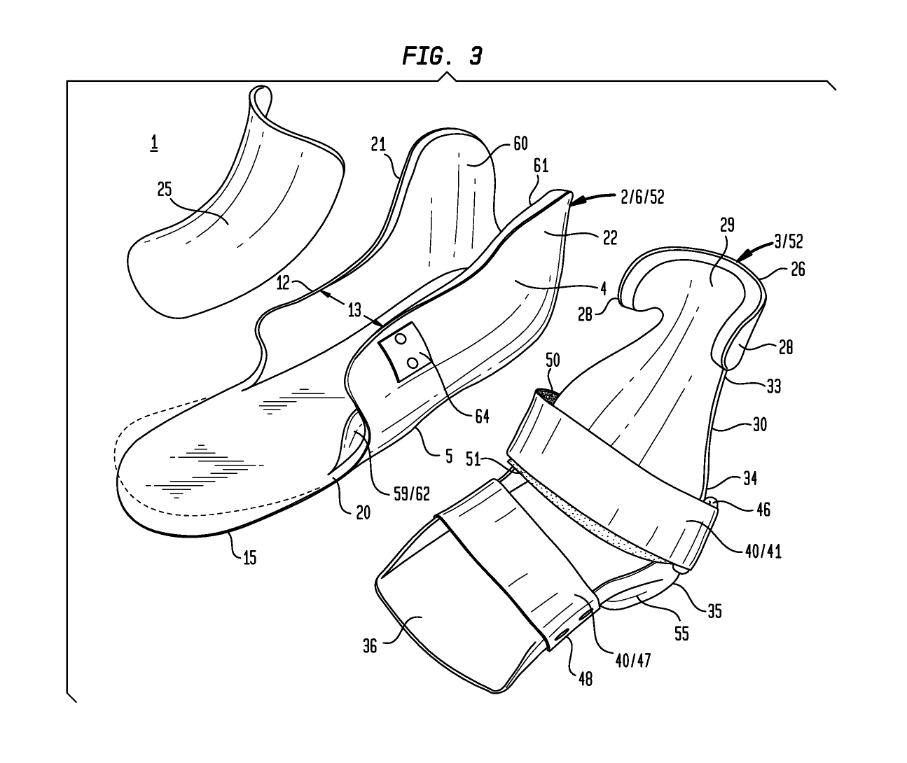

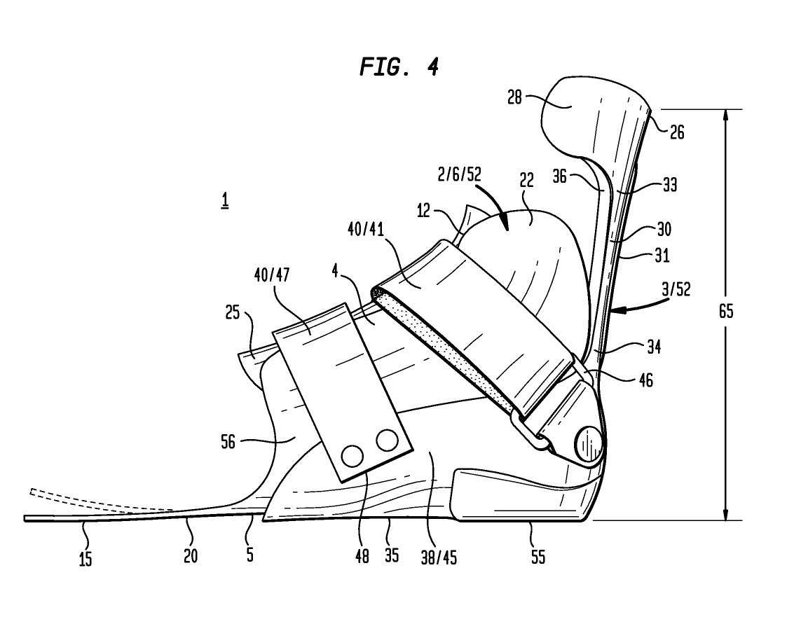

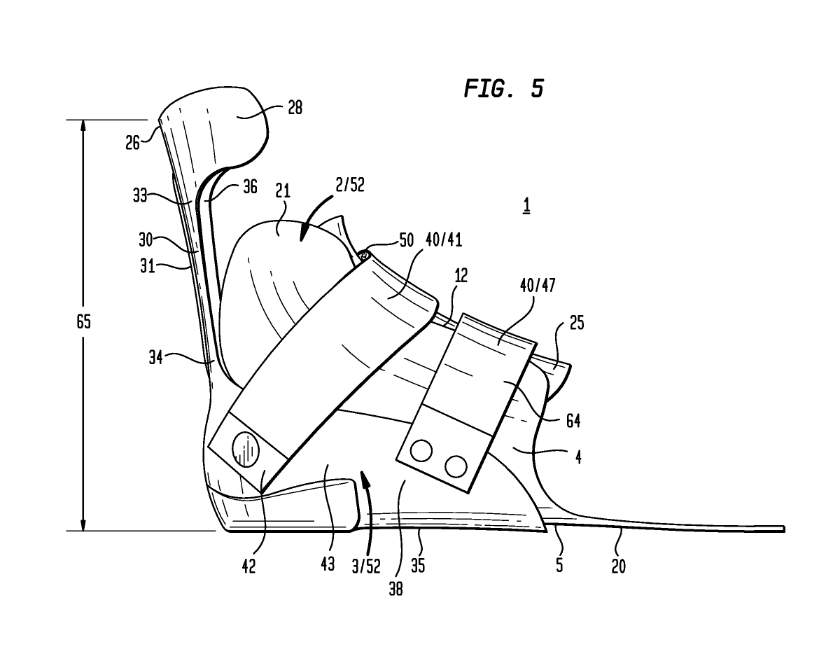

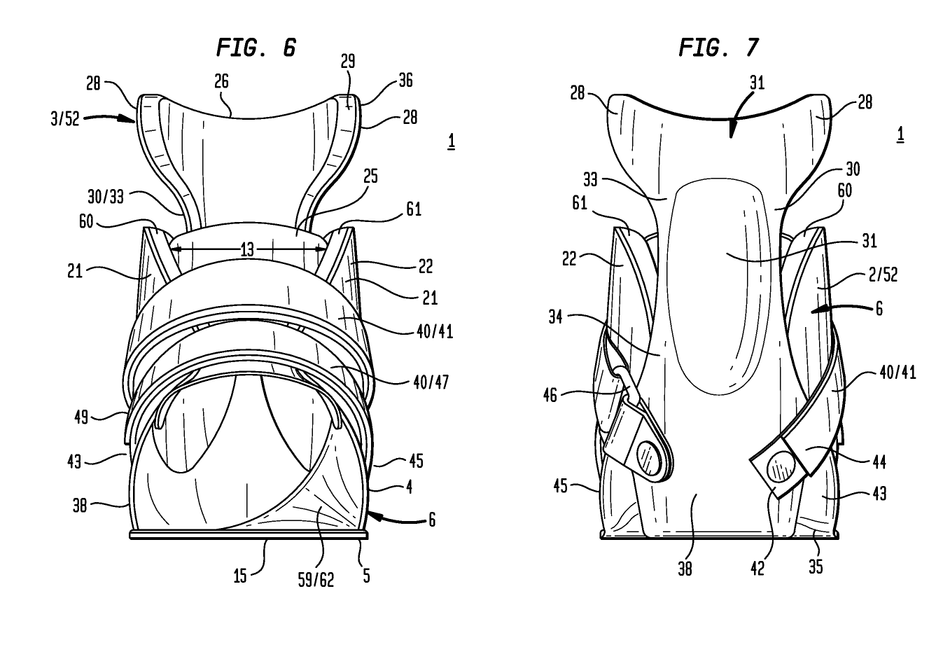

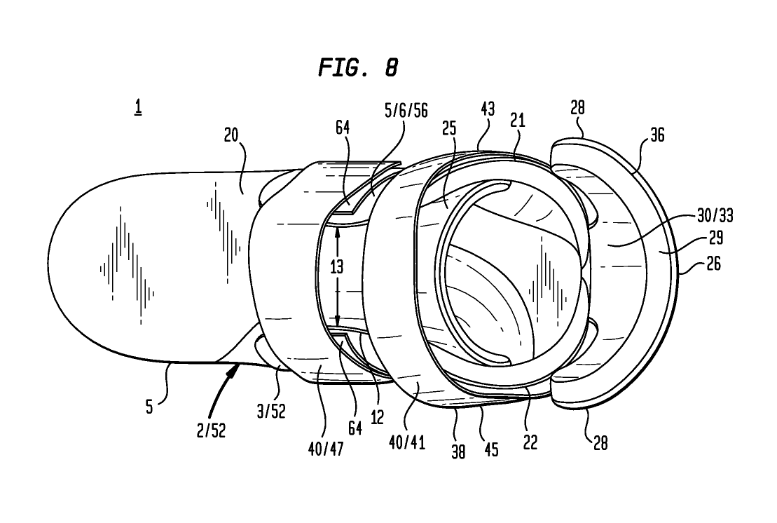

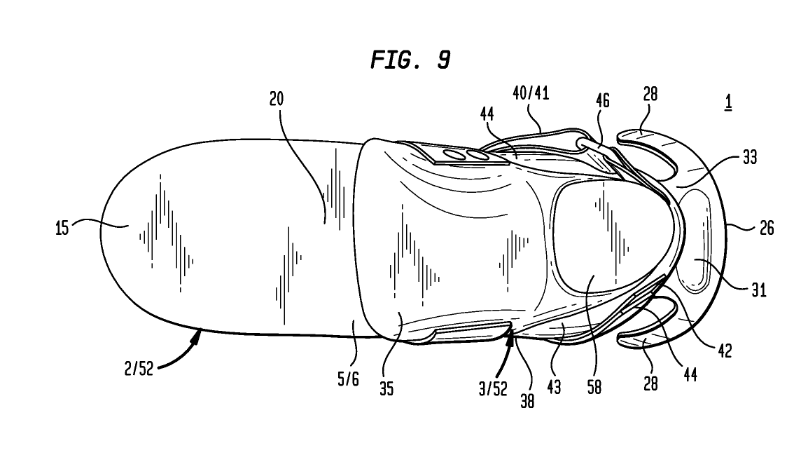

Generally referring to FIGS. 1 through 9 embodiments of the inventive orthosis (1) include two sections: a plantar section (2) and a posterior leg section (3). As to particular embodiments, the plantar section (2) and the posterior leg section (3) comprise a unitary or one-piece orthosis (1)(as shown in the illustrative example of FIG. 2). As to other embodiments, the plantar section (2) can removably nest within the posterior leg section (3)(as shown in the illustrative example of FIG. 3) or the posterior leg section (3) can removably nest within the plantar section (2) in similar fashion to FIG. 1 depending upon the particular application.

The plantar section (2) includes an upper (4) secured to an outsole (5) configured in a combination (6) to receive within a foot (7) of a wearer (8) including the heel (9) of the foot (7) to about midfoot (9) of the foot (7)(as shown in the example of FIG. 3). The upper (4) and outsole (5) can comprise a unitary or one-piece combination (6) (as shown in the example of FIG. 3 and further described below). The upper (4) extends upward from the outsole (5) and intimately wraps around the heel (10) and the ankle (11) of the wearer (8) to terminate at a topline (12) disposed in opposed relation a distance (13) apart over a dorsum (14) of the foot (7) of the wearer (8)(as shown in the example of FIG. 1). A forefoot outsole (15) adapted to underlie the planum (16) of the forefoot (17) engages the sole (18) of a forefoot (17) of the wearer (8) can be coupled to the outsole (5) and extend from about the midfoot (9) toward or beyond the toes (19). The forefoot outsole (15) can include a flex element (20) or sufficiently resiliently flex to allow flexion of the toes (19) of the foot (7) received within the orthosis (1). The plantar section (2) can further include a pair of malleolus overlay elements (21) and (22) coupled to the upper (4) and configured to correspondingly overlay a medial malleolus (23) and a lateral malleolus (24) of the wearer (8). A dorsum pad (25) configured to underlay the upper (4) overlaying the dorsum (14) of the foot (7) can be included to span the distance (13) between the topline (12).

Embodiments of the inventive orthosis (1), whether configured as a unitary or one-piece embodiment or discrete plantar and posterior leg sections (2)(3), includes a posterior leg intercept element (26) configured to intercept the posterior lower leg (27) of the wearer (8) at a location above the ankle (11) over the achilles tendon or above the ankle (11) and below the soleus muscle. The posterior leg intercept element (26) can further include at least one lateral intercept element (28) extending laterally in radial relation about the posterior lower leg (27) of the wearer (8). A posterior intercept pad (29) can be coupled to the posterior intercept element (26). The posterior intercept pad (29) resiliently deforms on engagement with the posterior lower leg (27) of the wearer (8).

A shank element (30) upwardly extends in relation to the plantar section (2) and supports the posterior leg intercept element (26) at the location above the ankle (11) to intercept the posterior lower leg (27) of the wearer (8). The shank element (30) can further include a shank reinforcement element (31) configured to limit movement of the posterior leg intercept element (26) in relation to the plantar section (2) upon engagement with the posterior lower leg (27) of the wearer (8). Particular embodiments of the shank element (30) can further include a shank height adjustment element (31) operable to adjust height of the posterior leg intercept element (28) in relation to the plantar section (2).

Now referring primarily to FIG. 3, as to those embodiments which provide the plantar section (2) discrete from the posterior leg section (3), the posterior leg intercept element (26) can be a unitary part of the posterior leg section (3) and removably coupled to the plantar section (2), as above described. As to the illustrative embodiment shown in the FIGS. 1 and 3 through 9, embodiments of a unitary posterior leg section (3) can include the posterior leg intercept element (26) supported by the shank element (30) at a first shank end (33) and coupled by a second shank end (34) to a heel outsole (35). The unitary posterior leg section (3) having a posterior leg section internal surface (36) can be configured to receive in nested engagement a corresponding portion of the outsole (5) and a corresponding portion of the upper (4) of the plantar section (2) from the heel (10) to about midfoot (9).

The two sections (2) and (3) of the orthosis (1), whether unitary or as discrete elements, can be produced from one or more thin-sheeted materials (52) contoured to intimately follow the posterior lower leg (27) and foot (7) outline of the wearer (8). The orthosis (1) can be worn about or under a covering (37) for the foot (7) such as a stocking, sock, hose, or the like; and the orthosis external surface (38) whether unitary or discrete elements in nested engagement can be configured for location within normal footwear (39) such as shoes, sandles, or the like.

A restraint element (40) operable in relation to the plantar section (2) retains the foot (7) within the combination (6). The combination (6) upon operation of the retention element (38) limits flexion of the foot (7) at the ankle (11) of the wearer (8). The orthosis (1) by limiting flexion of the foot (7) at the ankle (11), correspondingly limits the ability of the wearer (8) to walk on the toes (19) of the foot (7). As to the illustrative embodiments of the orthosis (1) shown in the Figures, the retention element (40) can, as one example, include a first strap (41) having a first strap first end (42) attached at a first side (43) of the plantar section (2) (whether or not the two sections (2)(3) of the orthosis (1) are unitary or discrete elements in nested engagement), and a first strap second end (44) being a free end adapted to loop around a ring (46) coupled at a second side (45) of the plantar section (2). The first strap second end (44) passed though the ring (46) and drawn toward the first side (43) of the plantar section (2) adjusts the length of the first strap (41) between the first side (43) and the ring (46) at the second side (45) of the plantar section (2) to reduce the distance (13) which separates the topline (12) of the upper (4) proximate the ankle (11) of the wearer (7) (as shown in the example of FIG. 1). The first strap second end (44) drawn toward the first side (43) can be secured at the first side (43) of the plantar section (2) to secure the combination (6) about the ankle (11). The restraint element (40) can further include a second strap (47) having a second strap first end (48) attached at the first side (41) of the plantar section (2) and having a second strap second end (49) being a free end which can be drawn to overlay the dorsum (14) of the foot (7) and secured at the second side of the plantar section (2) to reduce the distance (13) which separates the topline (12) of the upper (4) overlaying the dorsum (14) of the foot (7). Each of the first strap (41) and the second strap (47) can secured by mated engagement of a hook material (50) and a loop material (51) such as VELCRO®.

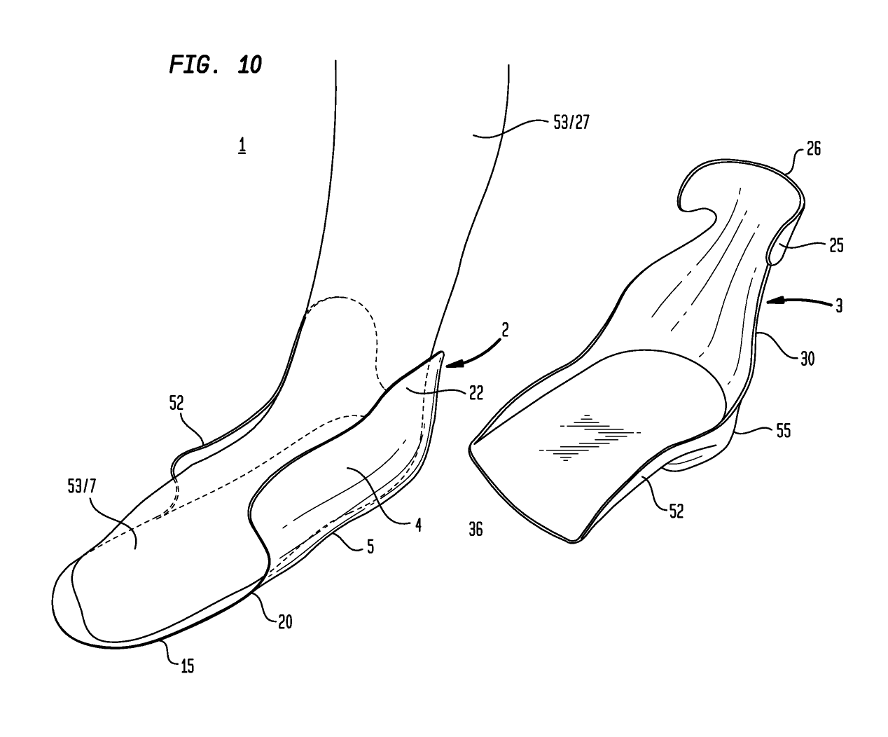

Now referring primarily to FIG. 10, the plantar section (2) of the orthosis (1) can be produced from one or more thin-sheet materials, composite materials, castable materials, or the like (collectively “orthosis material (52)”. The orthosis material (52) can as illustrative examples include: leather; plastics such as polypropylene, polyethylene, polyvinylchloride, castable thermoset plastics; composite materials including layers of laminatable material which can include fibers which can be the same, similar, or different in the same piece or between pieces of laminatable material such as boron carbide, silicon carbide, alumina, alumina titanium, carbon, KEVLAR®, INEGRA®, DYNEEMA®, SPECTRA®, s-glass, e-glass, or the like, which can be impregnated with an amount of resin such as: phenolic, epoxy, polyethylene terephtalate, vinylester, polyimides, bis(maleimide/diallybisphenol A, cyanate esters, thermoplastics, polypropyelene, nylon, or the like, to bond the layers together under pressure.

Again referring primarily to FIG. 10, an illustrative method of producing the plantar section (2) and the posterior leg section (3) includes a step in which the orthosis material (52) can be conformed or cast about a replica (53) of the posterior lower leg (27) and foot (7) of the wearer (8), such as a plaster cast or as a three dimensional model generated by a three dimensional scanner. As to particular embodiments, the plantar section (2) can be made by securing a discrete upper (2) to a discrete outsole (3) to produce the combination (6). As to other embodiments, the plantar section (2) can be made in one-piece by drawing thin sheet-material (5) (such as one or more pre-heated sheets of plastic or impregnated layers of laminatable material) about the replica (53) of the posterior lower leg (27) and foot (7). The thin sheet material (52) upon cooling or curing can be removed from the replica (53) of the posterior lower leg (27) and foot (7). The upper (4) of the combination (6) can be trimmed to produce the topline (12) of the combination (6) including, as to particular embodiments, the pair of malleolus overlay elements (21) and (22). The outsole (5) can be trimmed to produce embodiments which extend to the midfoot (9) or further extend to the forefoot (17) providing the forefoot outsole (15).

The posterior leg section (3), as to those embodiments which are one-piece with the plantar section (2), can be produced as above described, by extending the thin sheet material (52) (such as plastic sheet or laminatable materials) about of the portion of the replica (53) corresponding to the posterior lower leg (27) of the wearer (8). The shank reinforcement element (31) can be coupled to the portion of the thin sheet materials (52) corresponding to the shank element (30) of the orthosis (1). The shank reinforcement element (31) can be made from a thin elongate member of metal, plastic, or composite material sufficiently rigid to resist flexure of the shank element (30) upon engagement of the posterior lower leg (27) of the wearer (8). The cooled or cured unitary posterior leg section (3) and plantar section (2) can be trimmed to provide and adjustably locate the posterior leg intercept element (26) to engage a posterior lower leg (27) of the wearer (8) above the ankle (11) an below the soleus muscle over the achilles tendon and can further include at least one lateral intercept element (28) which extends from the posterior leg intercept element (26) in radial relation about the posterior lower leg (27) of the wearer (8).

As to those embodiments in which the posterior leg section (3) and the plantar section (2) are produced as discrete elements, as shown in the example of FIGS. 1 and 3 through 8, the plantar section (2) can be produced as above described from a first layer (54) of thin-sheeted material (52), and as to particular embodiments, a heel element (55) can be coupled to the combination external surface (56) at a location to correspondingly underlie the heel (10) of the foot (7). The untrimmed combination (6) including the heel element (55) can remain in conformed engagement to the replica (53). A release layer (57) can be applied to the combination external surface (56) of the untrimmed combination (6) conformingly engaged to the replica (53). The release layer (57) can be of any type of material which can be engaged to the combination external surface (56) of the untrimmed combination (6) and subsequently removed from the combination (6). The release layer (57) can be selected depending on the composition of the first layer (54) of thin-sheeted material (52). As non-limiting examples, the release layer (57) can include: TEFLON®, PEAL-PLYS®, silicon, latex, rubber, or the like. A second layer (58) of thin-sheeted material (53) (whether pre-heated plastic or impregnated laminatable materials) can be applied to the release layer (57) and conformed to the combination external surface (56) of the untrimmed combination (6). Upon subsequent cooling or curing, the second layer (58) corresponding to the untrimmed posterior leg section (3) can be released from the first layer (54) corresponding to the untrimmed plantar section (3). The first layer (54) can be released from the replica (53) and each trimmed to correspondingly provide the plantar section (2) which can be received in nested engagement within the posterior leg section (3)(as shown in the example of FIGS. 1 and 3).

An insole (59) along with the intercept pad (29) and a medial malleolus and a lateral malleolus pads (60) and (61) can be produced from a resiliently compressible sheet material (62)(such as polyurethane foam, polyethylene foams, polyvinylchloride foams, or the like) and coupled to the combination internal surface (63) of the combination (6), typically, by application of an adherent (63) such as contact cement to the engaged surfaces of the insole (59) and the combination (6). The dorsum pad (25) can be produced from a resiliently compressible material (such as such as polyurethane foam, polyethylene foams, polyvinylchloride foams, or the like) pre-heated and conformed to the dorsum (14) of the replica (53). The dorsum pad (25) can be produced and remain discrete from the combination (6) and positioned by the wearer (8) to underlay the upper (4) or can be one-piece with the insole (59) underlying the upper (4) and adapted to overlay the dorsum (14) of the foot (7).

As to those embodiments, in which the plantar section (2) nests within a discrete posterior leg section (3), the first strap first end (42) can be coupled to a first side (43) of the posterior leg section (3) and a ring element (46) can be coupled on the second side (45) of the posterior leg section (3), each at a location correspondingly proximate the heel (10) of the foot (7), allowing the first strap second end (44) of the first strap (41) being the free end to pass through the ring element (46) and drawn back to adjustably overlay the plantar section (2) around the ankle (11). The overlaid portion of the first strap (41) can engage a hook material (50) with a loop material (51) to secure the first strap (41) in fixed relation to the plantar section (2) reducing the distance (13) between the topline (12) of the upper (4) about the ankle (11).

A second strap first end (48) can be coupled to a first side (43) of the posterior leg section (3) at location corresponding to proximate the midfoot (9) and a second strap second end (49) being the free end of the second strap (47) can be drawn over the plantar section (2) overlying the dorsum (14) of the foot (7) and adjustably secured to the second side (45) of the posterior leg section (2) at a location corresponding to proximate the midfoot (9). The second strap (47) can engage a hook material (50) with a loop material (51) to secure the second strap (7) in fixed relation to the plantar section (3) which reduces the distance (13) between the topline (12) of the upper (4) overlaying the dorsum (14) of the foot (7). As to particular embodiments, the plantar section (2) can be retained nested within the posterior leg section (3) by coupling a band (64) of hook material (50) to the combination external surface (56) of the upper (4) at a location which allows a loop material (51) coupled to the second strap (47) to fasten with the band (64) of hook material (50).

Now referring primarily to FIG. 1, which shows a particular method of utilizing the inventive orthosis (1). The method can include the step of inserting a part of a foot (7) from a heel (10) to about midfoot (9) within a plantar section (2) of the orthosis (1) including a combination (6) of an upper (4) secured to an outsole (5) with the upper (4) extending from the outsole (5) to terminate at a topline (12) disposed about an ankle (11) and disposed in opposed relation a distance (13) apart over a dorsum (14) of the foot (7). The method can further include engaging a sole (18) of a forefoot (17) with a forefoot outsole (15) extending from the outsole (5) toward the toes (19) and can further include disposing a dorsum pad (25) to overlay the dorsum (14) of the foot (7) underlaying the upper (4) to span the distance (13) between the topline (12) of the upper (4). As to particular embodiments, the method further includes locating a pair of malleolus overlay elements (21)(22) of the combination (6) to correspondingly overlay a medial malleolus (23) and a lateral malleolus (24) of the ankle (11) of the foot (7).

As to particular embodiments, the method further includes locating the plantar section (2) of the orthosis (1) in nested engagement within a posterior leg section (3) of the orthosis (1) and thereby disposing a posterior leg intercept element (26) at a location which engages a posterior lower leg (27) above the ankle (11). As to particular embodiments, the method can further include adjusting a height (65) of the posterior leg intercept element (26) to corresponding locate the posterior leg intercept element (26) in relation to the posterior lower leg (27) above the ankle (11).

With the plantar section (2) nested within the lower leg portion (3), the method further includes operating a restraint element (40) which acts on the combination (6) of the upper (4) and the outsole (5) to retain the foot (7) within the combination (6) to limit flexion of the foot (7) at the ankle (11). The method further includes walking with the foot (7) retained within the inventive orthosis (1). As to particular embodiments of the inventive orthosis (1), the method further includes disposing the orthosis (1) retaining the foot (7) within footwear (39), such as a shoe.

As can be easily understood from the foregoing, the basic concepts of the present invention may be embodied in a variety of ways. The invention involves numerous and varied embodiments of an orthosis worn to limit toe walking and methods for making and using such orthosis including the best mode.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “dorsum pad” should be understood to encompass disclosure of the act of “dorsum padding” -- whether explicitly discussed or not -- and, conversely, were there effectively disclosure of the act of “dorsum padding”, such a disclosure should be understood to encompass disclosure of a “dorsum pad” and even a “means for dorsum padding.” Such alternative terms for each element or step are to be understood to be explicitly included in the description.

In addition, as to each term used it should be understood that unless its utilization in this application is inconsistent with such interpretation, common dictionary definitions should be understood to included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each orthosis herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in- part, or similar application.

1. An orthosis, comprising:
an upper secured to an outsole configured in a combination to receive within a foot of a wearer from a heel to about a midfoot, said upper extending from said outsole to terminate at a topline disposed in opposed relation a distance apart over a dorsum of said foot;
a retention element operable in relation to said combination to retain said foot within said combination, said combination upon operation of said retention element limits flexion of said foot at an ankle of said wearer; and
a posterior intercept element coupled to said combination, said posterior intercept element configured to intercept a posterior lower leg of said wearer at a location over the achilles tendon.

2. The orthosis of claim 1, further comprising a forefoot outsole coupled to said outsole, said forefoot outsole extending a distance beyond said midfoot adapted to engage a sole of a forefoot of said wearer, said forefoot outsole having a flex element which resiliently flexes to allow flexion of toes of said foot.

3. The orthosis of claim 2, wherein said upper, said outsole and said forefoot outsole comprise one-piece.

4. The orthosis of claim 3, further comprising a pair of malleolus overlay elements coupled to said combination, said pair of malleolus overlay elements configured to correspondingly overlay a medial malleolus and a lateral malleolus of said wearer.

5. The orthosis of claim 4, further comprising a dorsum pad configured to underlay said upper to span said distance between said topline overlaying said dorsum of said foot.

6. The orthosis of claim 1, further comprising a shank which upwardly extends from said combination to support said posterior leg intercept element at said location above said ankle to intercept said posterior lower leg of said wearer.

7. The orthosis of claim 6, further comprising a shank reinforcement element coupled to said shank, said shank reinforcement element configured to limit movement of said posterior leg intercept element in relation to said combination upon engagement with said posterior lower leg of said wearer.

8. The orthosis of claim 7, further comprising a shank height adjustment element operable to adjust height of said posterior leg intercept element in relation to said combination.

9. The orthosis of claim 8, further comprising a posterior intercept pad coupled to said posterior intercept element, said posterior leg intercept pad resiliently deformable on engagement with said posterior lower leg of said wearer.

10. The orthosis of claim 9, further comprising at least one lateral intercept element which extends from said posterior intercept element in radial relation about the lower leg of said wearer.

11. The orthosis of claim 10, wherein said at least one lateral intercept elements comprises a pair of lateral intercept elements which extends from said posterior intercept element in opposed direction in radial relation about the lower leg of said wearer.

12. The orthosis of claim 11, wherein said posterior intercept element comprises a discrete posterior intercept element which removably couples to said combination.

13. The orthosis of claim 1, wherein said retention element comprises a first strap adjustable in length to reduce said distance which separates said topline of said upper proximate said ankle of said wearer.

14. The orthosis of claim 13, wherein said retention element comprises a second strap adjustable in length to reduce said distance which separates said topline of said upper in relation to said midfoot of said wearer.

15. The orthosis of claim 14, wherein said first strap and said second strap connected to said discrete posterior intercept element, said first strap and said second strap operable to couple said posterior intercept element to said combination and retain said foot limiting flexion of said foot at an ankle of said wearer.

16. The orthosis of claim 12, further comprising an external surface of said orthosis configured for location within a shoe of said wearer.

17-40. (canceled)
